(19) United States
(12) Patent Application Publication
(10) Pub. No.: US 2025/0288229 A1
(43) Pub. Date: Sep. 18, 2025

(54) REMOTE SENSING MECHANISM DEVICE

(71) Applicant: Schott AG, Mainz (DE)

(72) Inventor: Andreas Koglbauer, Trebur (DE)

(73) Assignee: Schott AG, Mainz (DE)

(21) Appl. No.: 18/861,446

(22) PCT Filed: Mar. 23, 2023
(86) PCT No.: PCT/EP2023/057515

§ 371 Date: Oct. 29, 2024

(30) Foreign Application Priority Data
Apr. 29, 2022 (DE) 10 2022 110 489.3

(51) Int. Cl. A61B 5/1455

(52) U.S. Cl. CPC A61B 5/14552

(57) ABSTRACT

The invention relates to a remote sensing mechanism device comprising a primary light source for emitting primary light at a specific wavelength, an optical waveguide having a proximal end and a distal end and configured to transfer the primary light from the proximal end to the distal end and to transfer secondary light, caused at the distal end by the primary light and preferably at a different wavelength, back to the proximal end, a light receiver/output unit arranged at the distal end of the optical waveguide and serving to receive the primary light from the distal end and output the secondary light to the distal end of the optical waveguide, and a secondary light receiver arranged at the proximal end of the optical waveguide and serving to receive the secondary light from the proximal end of the optical waveguide, the optical waveguide having a numerical aperture of greater than 0.5.

### CROSS-REFERENCE TO RELATED APPLICATIONS

This application is a national stage entry under 35 U.S.C. § 371 of International Patent Application No. PCT/EP2023/057515 entitled “REMOTE SENSING MECHANISM DEVICE,” filed on Mar. 23, 2023, which is incorporated in its entirety herein by reference. International Patent Application No. PCT/EP2023/057515 claims priority to German Patent Application No. 10 2022 110 489.3 filed on Apr. 29, 2022, which is incorporated in its entirety herein by reference.

### BACKGROUND OF THE INVENTION

### 1. Technical Field of the Invention

The invention relates to a remote sensing device comprising an optical waveguide having a proximal end and a distal end, a primary light source arranged at the proximal end and a unit arranged at the distal end for absorption of the primary light and emission of secondary light for return transmission to the proximal end. A light absorption/emission unit, or sensor unit, located at the distal end may in principle be used for or contribute to the detection of various measurement variables. For example, measurement of magnetic fields, conductivities, temperatures or oxygen saturations may be made possible or assisted.

### 2. Description of the Related Art

Depending on the area of application, various requirements may be placed on the optical waveguide. To this end, for example, diameters or materials of the light-guiding cores or surrounding cladding layers, or properties of a bundle, may be selected suitably, for example a bundle of individual fibers or tubes with different refractive indices. Depending on the field of application, certain constraints also need to be taken into account, for example flexibility and dimensions, miniaturization often being desirable in some fields, particularly in medical applications.

It is an object of the present invention to provide a remote sensing device that enables optimization for the respective requirements for various distal light absorption/emission or sensor units, and in particular allows miniaturization.

### SUMMARY OF THE INVENTION

To this end, the invention discloses a remote sensing device having a primary light source, an optical waveguide and a light absorption/emission unit.

The primary light source is adapted for emission of primary light with a first wavelength. The optical waveguide has a proximal end and a distal end, and is adapted for transmission of the primary light from the proximal end to the distal end and/or for return transmission of secondary light with a second wavelength, which is induced at the distal end by the primary light, to the proximal end.

At the distal end of the optical waveguide, there is the light absorption/emission unit for absorption of the primary light, in particular from the distal end, and for emission of the secondary light onto the distal end of the optical waveguide.

At the proximal end of the optical waveguide, there is also preferably a secondary light receiver for reception of the back-transmitted secondary light from the proximal end of the optical waveguide. The secondary light receiver is in particular a detector for the secondary light, and may for example be configured as a photodiode or, for example, as an imaging flat detector.

In particular, the optical waveguide has a numerical aperture that is greater than 0.4 or preferentially is greater than 0.5 or is greater than 0.6.

In this way, a comparatively high light collection efficiency may advantageously be made possible, sometimes together with small dimensions, which for various areas of application enables optimization of the requirements placed on the light absorption/emission or sensor unit.

The numerical aperture (NA) of the optical waveguide refers in the context of this disclosure to the sine of the acceptance angle x of the optical waveguide, i.e. the sine of the half aperture angle of the optical waveguide. This corresponds to the formula NA=n sin α for n=1. The specification that the optical waveguide has in particular a numerical aperture that is greater than 0.4, or preferentially is greater than 0.5 or is greater than 0.6, therefore corresponds to the specification that the optical waveguide has an acceptance angle α that is greater than 23.6° or preferentially is greater than 30.0° or is greater than 36.9°.

For the case that the optical waveguide comprises at least one glass fiber having a core and a cladding, in particular a step index fiber, the numerical aperture may for example also be specified as NA=\sqrt{n_{core}²−n_{cladding}²}, where n_{core} denotes the refractive index of the core and n_{cladding} denotes the refractive index of the cladding.

As described, the primary light comprises at least one first wavelength and the secondary light comprises at least one second wavelength. The second wavelength is, in particular, a wavelength different to the first wavelength. In respect of particular embodiments, however, it is not intended to be ruled out that the first and the second wavelengths are identical. The primary light as well as the second light may of course also be formed as a spectrum.

The light absorption/emission unit is in particular configured as a sensor unit and/or interaction unit, the light absorption/emission unit emitting the secondary light after or during the irradiation of the primary light, and in this context enabling determination of a measurement variable. It is, however, not intended to be ruled out that an interaction of the primary light also takes place outside the light absorption/emission unit, for example in a tissue to be examined. To this extent, in one embodiment, the light absorption/emission unit may also be configured to absorb secondary light generated outside the device and in turn to emit this light onto the distal end of the optical waveguide, or optionally also to emit it as tertiary light onto the distal end of the optical waveguide after a further process.

According to one embodiment, the optical waveguide may be configured as a so-called Anderson waveguide or TAL waveguide. In particular, the optical waveguide may be adapted to transmit the primary light and/or the secondary light in a transversely localized fashion, in particular to transmit it with a transverse spatial resolution, in which case the optical waveguide may also be configured as an image guide.

Transversely localized transmission has in particular the advantage that less excitation light, or primary light, is necessary, a lower stray light background may be made possible, and/or minimization of the unused excitation light, or primary light, which may otherwise possibly be emitted into the sample space, is made possible. In particular, excitation with primary light on a sometimes small area is therefore made possible, while on the other hand absorption of secondary light may take place over a relatively large cross section.

The optical waveguide may in particular comprise a multiplicity of structure elements, each of which extends from the proximal end to the distal end and in part over the cross section of the optical waveguide, in such a way that a multiplicity of cross-sectional regions are defined in the cross section of the waveguide, each of which corresponds to the cross section of a single structure element.

Structure elements, in particular their cross-sectional regions, are preferably arranged nonuniformly in such a way that a transverse Anderson localization of the primary light and/or of the secondary light is induced.

According to one embodiment, the light absorption/emission unit at the distal end of the optical waveguide comprises a material that enables emission of the secondary light by absorption of the primary light. For example, the material may enable coherent processes, for example frequency doubling.

In particular, the light absorption/emission unit at the distal end of the optical waveguide comprises an excitable material which has an electronic structure that enables excitation by the primary light and decay by emission of the secondary light.

The excitation may preferably be enabled by primary light with a wavelength of between 200 nm and 20 μm.

Alternatively or in addition, the decay may preferably be enabled by emission of secondary light with a wavelength of between 200 nm and 20 μm.

The energy states are preferably configured in such a way that an external measurement variable can be measured with the aid of the received secondary light, for example an external measurement variable from the group consisting of a magnetic field, a conductivity, a temperature, an amount of substance or substance concentration, for example an oxygen saturation.

According to one embodiment, the light absorption/emission unit at the distal end of the optical waveguide comprises a diamond with one or more nitrogen-vacancy centers as excitable material, which has an electronic structure that enables excitation by the primary light and decay by emission of the secondary light.

The excitation may preferably be enabled by primary light with a wavelength of between 500 nm and 560 nm, for example 532 nm. Alternatively or in addition, the decay may preferably be enabled by emission of secondary light with a wavelength of between 600 nm and 800 nm.

The energy states are preferably configured in such a way that an external magnetic field can be measured with the aid of the received secondary light, in particular with the aid of splitting and/or an energy shift of spectral lines under the influence of the external magnetic field, preferentially by the irradiation of microwaves.

The light absorption/emission unit, in particular the diamond, may also comprise further or other centers, in particular as excitable material, for example one or more elements of the carbon-silicon group (the fourth main group). For example, the light absorption/emission unit, in particular the diamond, may comprise one or more of the following elements: Si, Ge, Sn, Pb.

The excitation may, particularly in the case of Si, preferably also take place by primary light with a wavelength of between 708 nm and 768 nm. The excitation may, particularly in the case of Ge, preferably also take place by primary light with a wavelength of between 572 nm and 632 nm. The excitation may, particularly in the case of Sn, preferably also take place by primary light with a wavelength of between 590 nm and 650 nm. The excitation may, particularly in the case of Pb, preferably also take place by primary light with a wavelength of between 490 nm and 550 nm and/or between 522 nm and 582 nm.

Preferably, the optical waveguide has a low intrinsic fluorescence at the wavelength of the primary light and/or of the secondary light.

In one development, excitation with primary light comprising more than one wavelength, in particular comprising a spectrum, may also be provided.

The optical waveguide preferably has a high average refractive index in order to increase the critical angle at the interface with the light absorption/emission unit, in particular the diamond (n=2.4).

In one development of the invention, spatially restricted absorption of the primary light may be provided, in particular when the primary light is transmitted in a transversely localized fashion. In this way, for example, a gradient field measurement may be made possible.

In particular, the light absorption/emission unit, the nitrogen-vacancy center or centers and/or the excitable material may be arranged at the distal end of the optical waveguide in such a way that spatially restricted absorption of the primary light, in particular spatially restricted excitation by the primary light, is made possible when transversely localized transmission of primary light takes place through the optical waveguide.

The light absorption/emission unit, in particular the diamond, may also have a reflector for deviation of the primary light and/or of the secondary light, for example a chamfer and/or a coating, particularly in such a way that spatially restricted absorption of the primary light perpendicularly with respect to the cross-sectional area of the distal end of the optical waveguide is made possible. Furthermore, the reflector may be used to deviate the fraction of the secondary light that is not emitted in the direction of the distal end.

In one development, more complex optical configurations of the interaction zone may also be provided, for example lenses, a microlens array and/or parabolic mirror.

The light absorption/emission unit, in particular the diamond, may be mechanically connected to the distal end of the optical waveguide. In other words, the light absorption/emission unit, in particular the diamond, may be applied firmly on the distal end of the optical waveguide.

The light absorption/emission unit, in particular the diamond, preferably extends over at least 50% of the cross section of the distal end of the optical waveguide, particularly preferentially over at least 75% of the cross section of the distal end of the optical waveguide.

The nitrogen-vacancy centers or the excitable material are preferably arranged only in a spatial subregion of the light absorption/emission unit or of the diamond, for example in a radially inner subregion that is surrounded by a radially outer subregion without excitable material or without nitrogen-vacancy centers.

Preferably, a reflector for deviation of the primary light and/or of the secondary light, for example a chamfer and/or a coating, is provided in the radially outer subregion of the light absorption/emission unit or of the diamond.

The reflector may preferably deviate secondary light, in particular radially emitted secondary light, onto the distal end of the optical waveguide in order to increase the light collection efficiency of the optical waveguide. Alternatively or in addition, the reflector may also deviate primary light, in particular transversely localized primary light, onto the nitrogen-vacancy centers or the excitable material.

The light absorption/emission unit, in particular the diamond, the nitrogen-vacancy center or centers and/or the excitable material, may preferably be arranged at the distal end of the optical waveguide in such a way that at least 0.5%, preferably at least 5%, of the secondary light at the distal end can be coupled into the optical waveguide, in particular after deviation by the chamfer or the reflector.

The light absorption/emission unit, in particular the nitrogen-vacancy center or centers and/or the excitable material, may be arranged only over a subregion of the cross section of the distal end of the optical waveguide, preferably over a subregion of less than 50% of the cross-sectional area, particularly preferentially over a subregion of less than 25% of the cross-sectional area.

The optical waveguide may have a cross section of between 30 μm and 5000 μm, preferably between 50 μm and 3000 μm.

The optical waveguide may have a length of between 10 mm and 10 000 mm, preferably between 50 mm and 2000 mm.

The optical waveguide may be configured to be at least partially flexible and/or configured to be at least partially rigid or even semistiff.

In one development of the invention, a tapered optical waveguide may also be provided.

Further, in one development, the optical waveguide may have a cross section that is less than the cross section of the excitable material, particularly in order to avoid primary light traveling past the excitable material into the light absorption/emission unit.

Preferably, the optical waveguide has a transmission of at least 30%, preferentially at least 40%, more preferentially at least 50%, for a wavelength of 532 nm.

Further, the optical waveguide preferably has a transmission of at least 30%, preferentially at least 40%, more preferentially at least 50%, for a wavelength in the range of between 600 nm and 800 nm.

The optical waveguide may have an attenuation of less than 50 dB/m, in particular less than 10 dB/m, in particular less than 1 dB/m, for a wavelength of 532 nm and/or for a wavelength in the range of between 600 nm and 800 nm.

Preferentially, the optical waveguide is configured as a polarization-maintaining waveguide. The optical waveguide may be configured as a nonmagnetic waveguide.

The optical waveguide may comprise at least two different types of structure elements, namely a first type having a first refractive index and a second type having a second refractive index.

The difference between the refractive indices is preferably greater than 0.05, in particular greater than 0.1, in particular greater than 0.2, in particular greater than 0.5.

It may comprise a multiplicity of structure elements of the first type and a multiplicity of structure elements of the second type, the structure elements of the first type being configured as bodies, in particular rod-shaped or tubular bodies, comprising or consisting of a first medium, the first medium having the first refractive index, the structure elements of the second type being configured as bodies, in particular rod-shaped or tubular bodies, comprising or consisting of a second medium, the second medium having the second refractive index, or the structure elements of the second type being configured as cavities in the structure elements of the first type, the cavities preferably forming the second refractive index.

It may also comprise one structure element of the first type and a multiplicity of structure elements of the second type, the structure element of the first type being configured as a base body, in particular a monolithic base body, comprising or consisting of a first medium, the first medium having the first refractive index, and the structure elements of the second type being configured as cavities in the base body, the cavities preferably forming the second refractive index.

As already described, the structure elements, in particular their cross-sectional regions, may be arranged nonuniformly in order to induce a transverse Anderson localization of the primary light and/or of the secondary light. The structure elements may, for example, be arranged truly randomly. On the other hand, the nonuniformity may be defined by a predetermined rule.

For example, the structure elements, in particular their cross-sectional regions, may have a nonuniform arrangement which is defined uniquely by a predetermined rule, the nonuniform arrangement that is defined uniquely by the predetermined rule being configured
(a) as periodic positioning of structure elements, in particular their cross-sectional regions, the periodically positioned structure elements having a variation from one another which is configured nonuniformly but is defined uniquely by a predetermined rule,
the variation of the periodically positioned structure elements from one another preferably being configured as a variation of the type of the structure elements, of the refractive index of the structure elements and/or of the geometry (for example the shape, the diameter and/or the substructure) of the structure elements,
(b) as aperiodic positioning of structure elements, in particular their cross-sectional regions, the aperiodic positions of the structure elements being configured nonuniformly but defined uniquely by a predetermined rule,
the structure elements optionally also having a variation from one another which is configured nonuniformly but is defined uniquely by a predetermined rule,
and/or (c) as positioning of structure elements, in particular their cross-sectional regions, at periodic sites, some of the periodic sites being occupied and some of the periodic sites being unoccupied, and the occupation being configured defined uniquely by a predetermined rule,
the structure elements optionally also having a variation from one another which is configured nonuniformly but is defined uniquely by a predetermined rule.

In one embodiment of the invention, the remote sensing device comprises a microwave generator and/or a microwave antenna for irradiation of microwaves onto the light absorption/emission unit, in particular the diamond, the nitrogen-vacancy center or centers and/or the excitable material.

The remote sensing device preferably comprises an evaluation unit for evaluation of the secondary light received by the secondary light receiver in order to determine the external measurement variable with the aid of the received secondary light.

The invention further relates to a remote sensing unit having an optical waveguide and a light absorption/emission unit.

The optical waveguide has a proximal end and a distal end, and is adapted for transmission of primary light from the proximal end to the distal end and/or for return transmission of secondary light with a preferably different wavelength, which is induced at the distal end by the primary light, to the proximal end.

At the distal end of the optical waveguide, there is the light absorption/emission unit for absorption of the primary light, in particular from the distal end, and for emission of the secondary light onto the distal end of the optical waveguide.

In particular, the optical waveguide has a numerical aperture that is greater than 0.4 or preferentially is greater than 0.5 or is greater than 0.6.

The remote sensing unit may further comprise one or more of the features described above in connection with the remote sensing device.

Preferentially, the optical waveguide of the remote sensing unit and/or of the remote sensing device is configured to transmit the primary light from the proximal end to the distal end. Alternatively, however, the primary light may also reach the distal end and therefore the light absorption/emission unit in a different way, for instance by the primary light being guided as a free beam or via a different delivery fiber to the light absorption/emission unit at the distal end of the optical waveguide. The remote sensing unit and/or the remote sensing device may accordingly comprise a further delivery waveguide, which is configured to guide the primary light to the distal end of the optical waveguide.

The invention further relates to an endoscope comprising a remote sensing device or a remote sensing unit as described above.

### BRIEF DESCRIPTION OF THE DRAWINGS

Preferred exemplary embodiments of the invention will be described below with reference to the figures, in which:

FIG. 1: shows a schematic representation of the distal end of an optical waveguide with a light absorption/emission unit, which receives primary light in the axial direction, fitted on the distal end,

FIG. 2: shows a schematic representation of the distal end of an optical waveguide with a light absorption/emission unit, which receives primary light in the radial direction, mechanically fitted on the distal end,

FIGS. 3a-3f: show schematic illustrations of various possibilities for optical waveguides with structure elements, or their cross-sectional regions, configured nonuniformly but defined uniquely by a predetermined rule,

FIG. 4: shows a schematic illustration of various aspects for variations among structure elements, or their cross-sectional regions, and possibilities for combinations of these aspects,

FIGS. 5a-5b: show schematic illustrations of exemplary possibilities for optical waveguides with structure elements, or their cross-sectional regions, configured nonuniformly but defined uniquely by a predetermined rule, the waveguides each comprising one structure element of a first type and a multiplicity of structure elements of a second type, and optionally further types,

FIGS. 6a-6f: show schematic illustrations of various possibilities for waveguides with structure elements, or their cross-sectional regions, configured nonuniformly but defined uniquely by a predetermined rule, the waveguides each comprising a multiplicity of structure elements of a first type and a multiplicity of structure elements of a second type, and optionally further types,

FIG. 7: shows schematic perspective views of an optical waveguide with two types of structure elements, the cross-sectional regions of which are arranged nonuniformly distributed on a grid,

FIG. 8: shows a schematic representation of the distal end of an optical waveguide with a light absorption/emission unit, comprising excitable material which extends transversely over the entire width of the optical waveguide, fitted on the distal end.

### DETAILED DESCRIPTION OF THE INVENTION

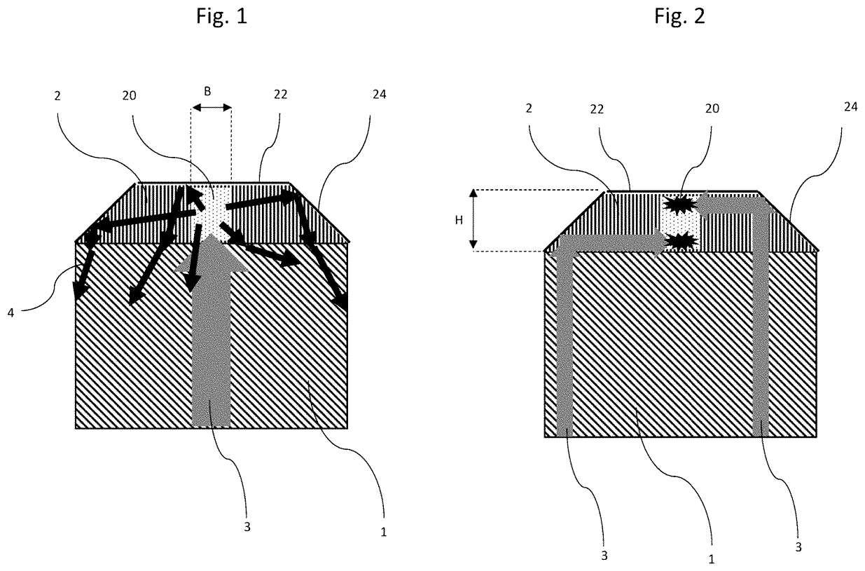

FIG. 1 shows the distal end of an optical waveguide 1 on which a light absorption/emission unit 2 is mechanically fitted. The light absorption/emission unit 2 comprises excitable material 20, which during operation receives the primary light 3 transmitted through the optical waveguide 1 and emits the secondary light 4.

In the example shown, the light absorption/emission unit 2 is configured as a diamond and the excitable material 20 is configured as a subregion of the diamond, in which there are one or more nitrogen-vacancy centers (NV centers). An NV center is characterized in that a carbon atom is replaced by a nitrogen atom (N) in the diamond lattice and a further neighboring carbon atom is missing (V).

The excitation takes place by primary light 3 with a wavelength of for example 532 nm, or a wavelength of between 515 nm and 550 nm. The secondary light 4 may have a wavelength of between 600 nm and 800 nm. The optical waveguide 1 is therefore preferably selected so that there is a transmission of at least 50%, preferably at least 70%, over the length of the optical waveguide 1 for a wavelength of 532 nm and respectively for a wavelength in the range of between 600 nm and 800 nm.

The NV centers are, or the excitable material 20 is, in this exemplary embodiment arranged in a locally restricted fashion in the light absorption/emission unit 2. In this example, the excitable material 20 is arranged transversely over a width B that is less than the width of the optical waveguide 1, in particular less than 40% or less than 30% of this width. The excitable material 20 is therefore to be encountered only in a locally restricted fashion transversely in the light absorption/emission unit 2, in this case the diamond.

Since the optical waveguide 1 is configured as an Anderson waveguide in this exemplary embodiment, the primary light 3 can be transmitted in a transversely localized fashion. It is thereby possible to excite the excitable material 20 arranged transversely only in a locally restricted fashion with primary light 3 transmitted in a transversely localized fashion, the transverse position of the primary light 3 corresponding to that of the excitable material 3. The reception of the primary light 3 by the excitable material takes place here in the axial direction.

The excitable material 20 emits the secondary light 4 in different directions, however, for example also in the radial direction. Because of the comparatively high numerical aperture of the optical waveguide 1, which in this case is greater than 0.5, a high light collection efficiency can be achieved in respect of the secondary light 4.

In order to further increase the light collection efficiency, the light absorption/emission unit 2, in this case the diamond, also has a reflective coating 22 that is applied on the outer surface. Further, the light absorption/emission unit 2, in this case the diamond, has a circumferential chamfer 24 for deviation of the secondary light, which may likewise be provided with the coating 22.

FIG. 2 shows the distal end of an optical waveguide 1 as in FIG. 1, although in this case the transverse position of the primary light 3 differs from that of the excitable material 20 arranged in a locally restricted fashion transversely. In particular, the transverse position of the primary light 3 may lie in the region of the reflector or of the circumferential chamfer 24 of the light absorption/emission unit 2. The excitable material 20 can therefore be excited in a spatially restricted fashion in the axial direction (i.e. perpendicularly with respect to the cross-sectional area of the distal end of the optical waveguide). It is accordingly possible that the excitable material 20 extending over the height H is excited only over a part of this height H, so that a gradient field measurement is made possible.

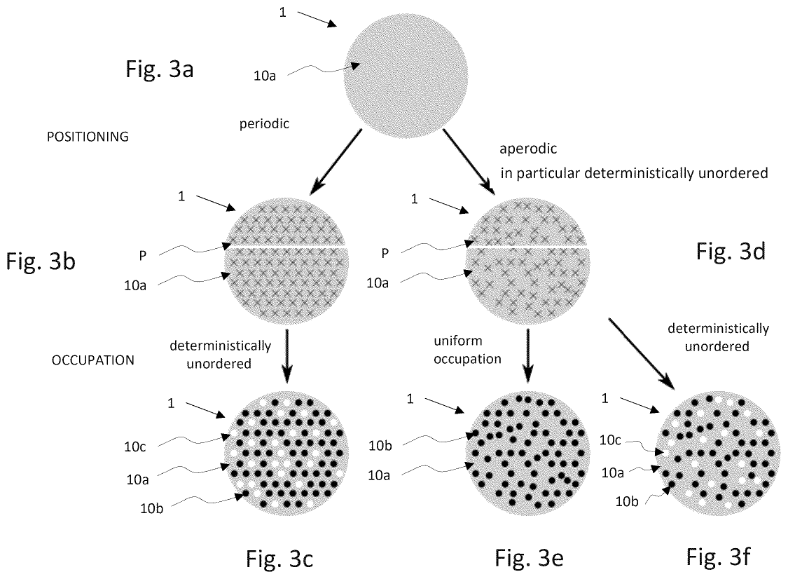

Various embodiments of a possible nonuniformity of the structure elements of the optical waveguide will be discussed below with reference to FIGS. 3a to 6f, again by way of example. As described, the structure elements, in particular their cross-sectional regions, may be characterized on the one hand by a nonuniformity in relation to one another, and on the other hand by a regularity insofar as the nonuniformity of the structure elements is uniquely predetermined, and in particular is deterministic and/or reproducible and is not governed by chance.

For example, the structure elements, or their cross-sectional regions, may have a nonuniform arrangement which is defined uniquely by a predetermined rule, have nonuniform geometries with respect to one another which are defined uniquely by a predetermined rule and/or have nonuniform refractive indices with respect to one another which are defined uniquely by the predetermined rule.

With the aid of a tree diagram, FIGS. 3a to 3f show various possibilities for producing a nonuniform arrangement which is defined uniquely by a predetermined rule. As the starting point, FIG. 3a shows a structure element 10a which, for example, may be configured as a matrix material (it is also possible for the structure element 10a to be formed as air, or to be absent). FIG. 3b shows a further starting point, derived therefrom, with the structure element 10a and a multiplicity of periodic positions P for occupation with structure elements, which then have periodic positioning. FIG. 3d shows a further starting point derived from FIG. 3a, with the structure element 10a and a multiplicity of aperiodic positions P for occupation with structure elements, in order to obtain aperiodic positioning. On the basis of the starting points shown in FIGS. 3b and 3d, occupation of the positions P with structure elements leads to waveguides according to the invention, as will be described in more detail below.

On the basis of FIG. 3b, FIG. 3c shows a waveguide 1 having structure elements 10b, 10c, the cross-sectional regions of which have aperiodic positioning and/or lie at periodic positions. The waveguide shown in FIG. 3c has three types of structure elements 10a, 10b, 10c, each of which may have a different refractive index. For example, the structure element 10a may be configured as a matrix material and the structure elements 10b and 10c may be cavities in the matrix material, which are filled with materials of different refractive indices.

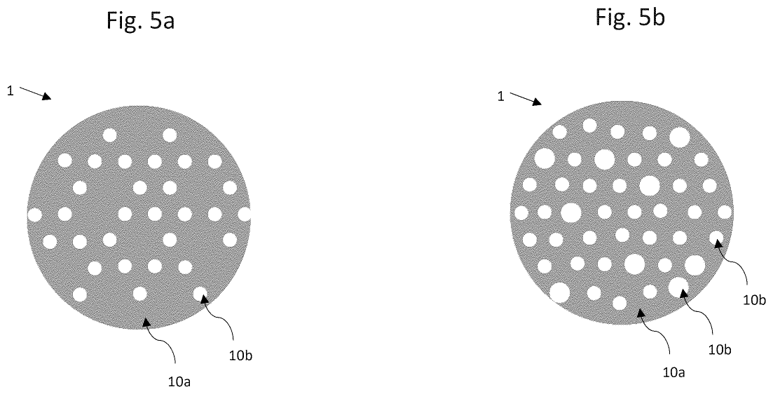

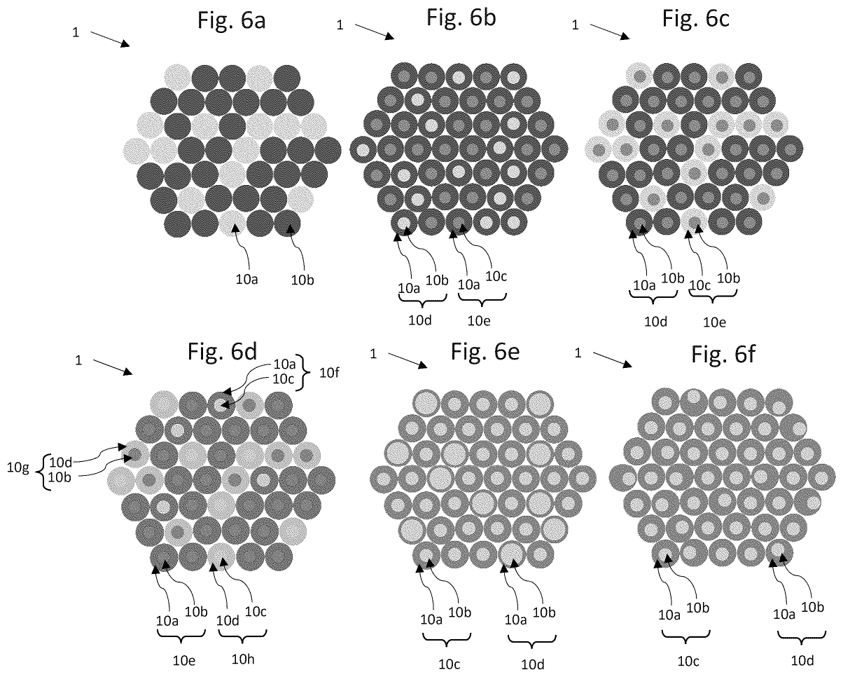

It is, however, likewise possible that one of the materials of the structure elements 10b and 10c in turn corresponds to the matrix material of the structure element 10a, or that the (filled) cavities corresponding to these structure elements in the matrix material are missing (cf. below in relation to FIG. 5a). It is likewise possible that the structure element 10a is formed as air, or is absent, and the structure elements 10b and 10c are adjacent to one another (cf. below in relation to FIG. 6a).

The waveguide 1 shown in FIG. 3c has structure elements 10b, 10c with periodic positioning. The structure elements 10b, 10c are of different types, however, and the occupation of the different types on the regular grid is nonuniform but defined by a predetermined rule. In particular, the variation of the structure elements 10b, 10c from one another is therefore nonuniform but defined by a predetermined rule. The structure elements 10b, 10c may, in particular, be referred to as deterministically unordered. FIG. 3c therefore shows a case of a waveguide 1 wherein the structure elements, or their cross-sectional regions, have a nonuniform arrangement which is defined uniquely by a predetermined rule. The term arrangement is to be understood here as meaning that the selection or occupation of the various types of structure elements 10b, 10c at the respective periodic positions is nonuniform but is defined by a predetermined rule, that is to say it is not random.

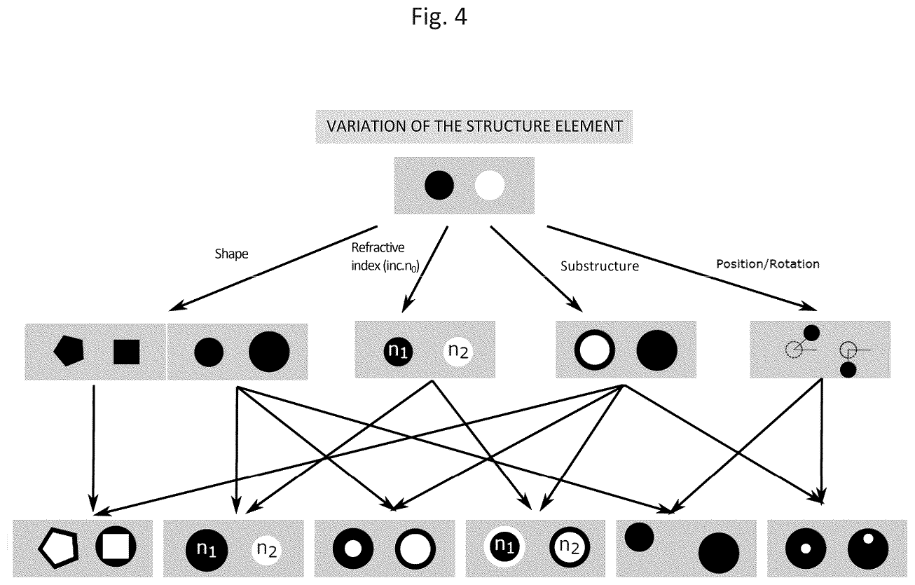

It is furthermore possible that the structure elements 10b, 10c do not differ in respect of their refractive indices, that is to say for example they have the same refractive index or consist of the same material, but vary in respect of other aspects (cf. below in relation to FIG. 4). It is further possible that the structure elements 10b, 10c differ both in respect of their refractive indices and in respect of other aspects.

On the basis of FIG. 3d, FIG. 3e shows a waveguide 1 having two types of structure elements, namely the structure element 10a, which may for example be configured as a matrix material, and a multiplicity of structure elements 10b which may for example be configured as cavities, in particular filled cavities, in the matrix material. The cross-sectional regions of the structure elements 10b are positioned aperiodically in this case. The positioning of the structure elements 10b may then constitute the nonuniformity which is defined by a predetermined rule. In particular, the structure elements 10b of the second type may have positions that are nonuniform but defined by a predetermined rule. FIG. 3e therefore shows a case of a waveguide 1 wherein the structure elements, or their cross-sectional regions, have a nonuniform arrangement which is defined uniquely by a predetermined rule. The term arrangement is to be understood here as meaning that the structure elements or some of the structure elements, or their cross-sectional regions, are positioned aperiodically, the positions being defined by the predetermined rule, that is to say they are not random. In the case of FIG. 3e, in particular, the structure elements 10b of the second type have a uniform refractive index, have uniform geometries and/or are configured uniformly in respect of further aspects, and in particular are configured identically. This case may be described as uniform occupation of the aperiodic positions.

Conversely, on the basis of FIG. 3d, FIG. 3f shows a waveguide 1 in which aperiodic positioning of structure elements is provided together with different types of structure elements 10b, 10c. In this case, the nonuniformity that is defined uniquely by a predetermined rule may consist in the aperiodic positioning of the structure elements 10b, 10c or in the occupation, that is to say the variation of the structure elements 10b, 10c from one another, or both in the positioning and in the occupation.

FIG. 4 shows various possibilities of variations that structure elements may have from one another (central row) and exemplary combination possibilities, which are not to be regarded as exhaustive, of the variations (bottom row). The variations shown may in particular be employed for occupation of positions with structure elements which is configured nonuniformly but is defined uniquely by a predetermined rule. Structure elements, the cross-sectional regions of which are localized at periodic or aperiodic positions, for example inside a matrix material, may for example vary from one another in respect of their shape, vary in respect of their type or refractive index, vary in respect of in respect of their substructure and/or vary in respect of their rotation (and/or local position).

For example, variations of the geometries of the structure elements, in particular their cross-sectional regions, may be configured as variations of the shape (number of vertices, diameter). Variations of the geometry may also be configured as variations of the substructure. A substructure may, in particular, consist in a structure element, in particular its cross-sectional region, having at least two different regions of different refractive indices, in particular a core and a surrounding cladding (core-cladding system).

In combination, for example, a first type of structure elements may have a polygonal cladding and/or a polygonal core and a second type of structure elements may have a round cladding and a polygonal core (bottom row, first column). These two types of structure elements may then, for example, be used to occupy periodic or alternatively aperiodic positions.

Further, for example, a first type of structure elements may have a first refractive index and a first diameter and a second type of structure elements may have a second refractive index and a second diameter (bottom row, second column); or a first type of structure elements may have a core-cladding system with a core having a first diameter and a second type of structure elements may have a core-cladding system with a core having a second diameter (bottom row, third column); or a first type of structure elements may have a core-cladding system with a core having a first refractive index and a second type of structure elements may have a core-cladding system with a core having a second refractive index (bottom row, fourth column); or a first type of structure elements may have a first diameter and a rotation about a point of rotation lying outside the structure element and a second type of type of structure elements may have a second diameter and a rotation about a point of rotation lying outside the structure element (bottom row, fifth column), or a first type of structure elements may have a core-cladding system with a centered core and a second type of structure elements may have a core-cladding system with a core having a rotation about a point of rotation lying outside the core (bottom row, sixth column), etc.

FIG. 5a shows a waveguide 1 which is respectively comparable in some aspects to the waveguide of FIG. 3c. The waveguide has a first structure element 10a, which may for example be configured as a matrix material. The waveguide further has a multiplicity of structure elements 10b which may for example be configured as filamented cavities in the matrix material. The structure elements 10b lie at periodic sites, but not all periodic sites are occupied with a structure element. FIG. 5a therefore shows a case of a waveguide 1 wherein the structure elements, or their cross-sectional regions, have a nonuniform arrangement which is defined uniquely by a predetermined rule. The term arrangement is to be understood here as meaning that the structure elements or some of the structure elements, or their cross-sectional regions, lie at periodic sites, some of the periodic sites being occupied and some of the periodic sites being unoccupied, and the occupation being configured defined uniquely by a predetermined rule, that is to say it is not random.

FIG. 5b shows a waveguide 1 which is respectively comparable in some aspects to the waveguide of FIG. 3f. The waveguide has a first structure element 10a, which may for example be configured as a matrix material. The waveguide further has a multiplicity of structure elements 10b with a first diameter and a multiplicity of structure elements 10c with a second diameter. The structure elements in this example are positioned aperiodically, in which case the aperiodic positioning may be configured nonuniformly but defined uniquely by a predetermined rule. FIG. 5b therefore shows a case of a waveguide 1 wherein the structure elements, or their cross-sectional regions, have a nonuniform arrangement which is defined uniquely by a predetermined rule. The term arrangement is to be understood here as meaning that the structure elements or some of the structure elements, or their cross-sectional regions, are positioned aperiodically, the aperiodic positions being defined by the predetermined rule, that is to say they are not random, and/or the structure elements having a variation from one another which is configured nonuniformly but is defined uniquely by a predetermined rule, the variation being configured as two types of structure elements, for example with different diameters.

FIGS. 6a to 6f show a few waveguides 1, each having a multiplicity of structure elements of a first type and a multiplicity of structure elements of a second type (and sometimes further types, in FIG. 6d). In particular, the waveguides 1 shown here do not have a matrix but instead the structure elements are adjacent to one another. A common feature of the waveguides 1 shown in FIGS. 6a to 6f is that the structure elements of the various types, in particular their cross-sectional regions, are positioned periodically but the occupation of the periodic positions with the types of structure elements is configured nonuniformly but defined uniquely by a predetermined rule. The waveguides 1 shown in FIGS. 6a to 6f are therefore distinguished in that the structure elements, or their cross-sectional regions, have a nonuniform arrangement which is defined uniquely by a predetermined rule, in which case the term arrangement is to be understood as meaning that the selection or occupation of the various types of structure elements at the periodic positions is nonuniform but is defined by the predetermined rule, that is to say it is not random.

FIG. 6a shows for instance a waveguide 1 having a multiplicity of structure elements 10a and a multiplicity of structure elements 10b, which have different refractive indices.

FIG. 6b shows a waveguide 1 having a multiplicity of structure elements 10d and a multiplicity of structure elements 10e, which have different refractive indices and a different substructure, the substructure being defined by the substructure elements 10a and 10b (with refractive indices a and b) and 10a and 10c (with refractive indices a and c) respectively. The substructure in this case consists in the structure elements 10d and 10e being configured as core-cladding systems, the cores being different.

FIG. 6c similarly shows a waveguide 1 having a multiplicity of structure elements 10d and a multiplicity of structure elements 10e, which have different refractive indices and a different substructure, the substructure being defined by the substructure elements 10a and 10b (with refractive indices a and b) and 10c and 10b (with refractive indices c and b) respectively. The substructure in this case consists in the structure elements 10d and 10e being configured as core-cladding systems, the claddings being different.

FIG. 6d similarly shows a waveguide 1 having a multiplicity of structure elements 10e, a multiplicity of structure elements 10f, a multiplicity of structure elements 10g and a multiplicity of structure elements 10h, which have different refractive indices and a different substructure, the substructure being defined by the substructure elements 10a and 10b (with refractive indices a and b) and 10a and 10c (with refractive indices a and c) and 10b and 10d (with refractive indices b and d) and 10c and 10d (with refractive indices c and d) respectively. The substructure in this case consists in the structure elements 10e, 10f, 10g and 10h being configured as core-cladding systems, both the claddings and the cores being different.

FIG. 6e shows a waveguide 1 having a multiplicity of structure elements 10c and a multiplicity of structure elements 10d, which have different geometries and a different substructure, the substructure of the structure element 10c being defined by the substructure elements 10a and 10b (with refractive indices a and b and a first core diameter), and the substructure of the structure element 10d being defined by the substructure elements 10a and 10b (with refractive indices a and b and a second core diameter).

FIG. 6f shows a waveguide 1 having a multiplicity of structure elements 10c and a multiplicity of structure elements 10d, which have different geometries and a different substructure, the substructure of the structure element 10c being defined by the substructure elements 10a and 10b (with refractive indices a and b and a centrally positioned core), and the substructure of the structure element 10d being defined by the substructure elements 10a and 10b (with refractive indices a and b and a core positioned off-center).

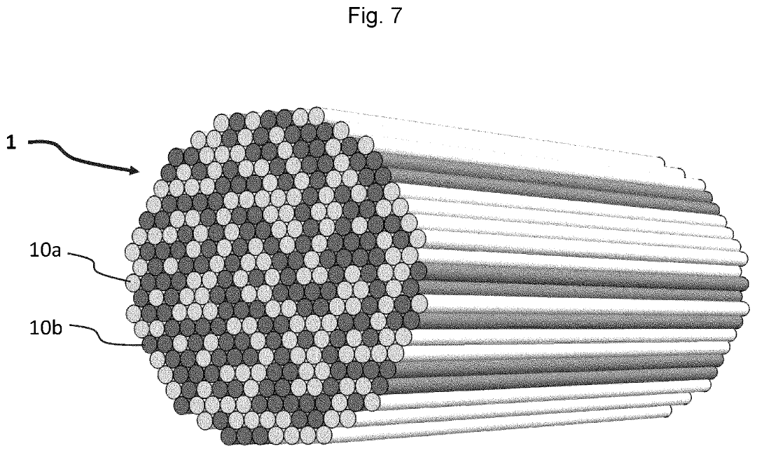

FIG. 7 shows by way of example a three-dimensional view of an optical waveguide 1 having a multiplicity of structure elements of a first type 10a and a multiplicity of structure elements of a second type 10b. In this example, the cross-sectional regions of the structure elements are arranged on a periodic grid.

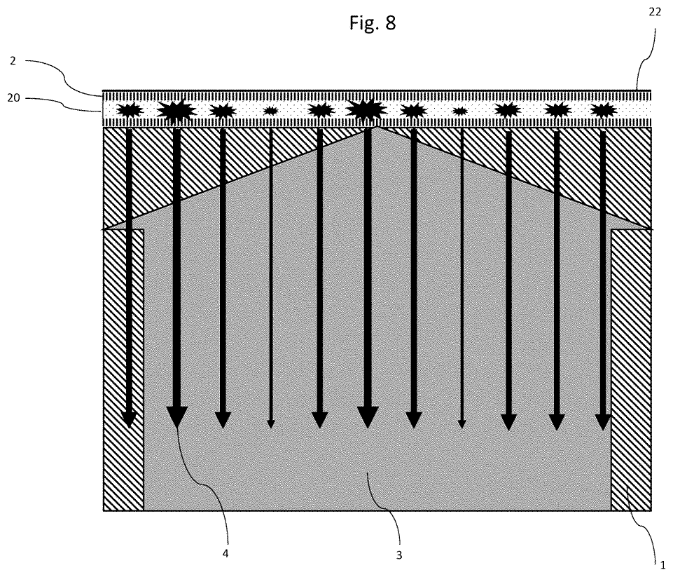

FIG. 8 shows a further exemplary embodiment of a remote sensing unit having an optical waveguide 1 and a light absorption/emission unit 2 fitted on the distal end of the optical waveguide 1. The light absorption/emission unit 2 in this example comprises excitable material 20 which extends in the radial direction substantially over the entire width of the optical waveguide. Alternatively, the excitable material may also extend over a width that corresponds to at least 50 percent, preferably at least 75 percent, of the width of the light absorption/emission unit 2 and/or of the optical waveguide 1. In some embodiments, the excitable material extends in the axial direction (i.e. perpendicularly with respect to the cross-sectional area of the distal end of the optical waveguide) only over a part of the light absorption/emission unit 2, for example over less than 90 percent, in particular over less than 75 percent of the height of the light absorption/emission unit 2. Preferentially, however, the excitable material extends in the axial direction over the entire light absorption/emission unit 2.

Specifically, the light absorption/emission unit 2 may for example be configured as a diamond layer which extends, for example, over the entire distal facet. The excitable material may, for example, be configured as a homogeneous (at least radially) occupation with NV centers.

In the exemplary embodiment described, surface-wide exposure of the light absorption/emission unit 2 and/or of the excitable material 20 to primary light 3 may in particular take place. The secondary light 4 emitted by the light absorption/emission unit 2 at the distal end of the optical waveguide 1 may have a different intensity in the radial direction, i.e. along the width of the excitable material 20. This secondary light 4 may then be transmitted in a transversely localized fashion through the optical waveguide 1, which is preferably in turn configured as an Anderson waveguide.

By the exemplary embodiment described, it is therefore possible to produce a remote sensing unit that forms a radially position-resolved sensor, for example a 2D sensor.

The German patent application DE 10 2020 116 444.0 and the international patent application PCT/EP2021/066986 are hereby incorporated by reference.

1-17. (canceled)

18. A remote sensing device, comprising:
a primary light source for emission of primary light with a first wavelength;
an optical waveguide having a proximal end and a distal end and adapted for transmission of the primary light from the proximal end to the distal end and for return transmission of secondary light with a second wavelength, which is induced at the distal end by the primary light, to the proximal end, the optical waveguide having a numerical aperture that is greater than 0.5;
a light absorption/emission unit arranged at the distal end of the optical waveguide for absorption of the primary light from the distal end and for emission of the secondary light onto the distal end of the optical waveguide; and
a secondary light receiver arranged at the proximal end of the optical waveguide for reception of the secondary light from the proximal end of the optical waveguide.

19. The remote sensing device of claim 18, wherein at least one of the following is satisfied:
the optical waveguide is adapted to transmit the primary light and/or the secondary light in a transversely localized fashion with a transverse spatial resolution in such a way that the optical waveguide forms an image guide;
the optical waveguide comprises a multiplicity of structure elements, each of which extends from the proximal end to the distal end and in part over a cross section of the optical waveguide, in such a way that a multiplicity of cross-sectional regions are defined in the cross section of the waveguide, each of which corresponds to the cross section of a single structure element; or
the cross-sectional regions of the structure elements are arranged nonuniformly in order to induce a transverse Anderson localization of the primary light and/or of the secondary light. 20 (New) The remote sensing device of claim 18, wherein the light absorption/emission unit at the distal end of the optical waveguide comprises a material that enables emission of the secondary light by absorption of the primary light and/or the light absorption/emission unit at the distal end of the optical waveguide comprises an excitable material which has an electronic structure that enables excitation by the primary light and decay by emission of the secondary light.

21. The remote sensing device of claim 20, wherein at least one of the following is satisfied:
the excitation is enabled by primary light with a wavelength of between 200 nm and 20 μm and/or the decay is enabled by emission of secondary light with a wavelength of between 200 nm and 20 μm; or
the energy states are configured in such a way that an external measurement variable can be measured with the aid of the received secondary light, wherein the external measurement variable is selected from the group consisting of a magnetic field, a conductivity, a temperature, and an amount of substance or substance concentration.

22. The remote sensing device of claim 18, wherein the light absorption/emission unit at the distal end of the optical waveguide comprises a diamond with one or more nitrogen-vacancy centers as excitable material, which has an electronic structure that enables excitation by the primary light and decay by emission of the secondary light.

23. The remote sensing device of claim 22, further comprising at least one of the following:
a microwave generator and/or a microwave antenna for irradiation of microwaves onto the diamond, the one or more nitrogen-vacancy centers, and/or the excitable material; or
an evaluation unit for evaluation of the secondary light received by the secondary light receiver in order to determine an external measurement variable with the aid of the received secondary light.

24. The remote sensing device of claim 22, wherein the one or more nitrogen-vacancy centers and/or the excitable material is or are arranged at the distal end of the optical waveguide in such a way that spatially restricted excitation by the primary light is made possible when transversely localized transmission of primary light takes place through the optical waveguide;
and/or wherein the diamond has a reflector for deviation of the primary light and/or of the secondary light in such a way that spatially restricted absorption of the primary light perpendicularly with respect to a cross-sectional area of the distal end of the optical waveguide is made possible.

25. The remote sensing device of claim 22, wherein the diamond is mechanically connected to the distal end of the optical waveguide.

26. The remote sensing device of claim 22, wherein the one or more nitrogen-vacancy centers or the excitable material radially extend substantially over an entire width of the diamond or extend at least over 50% of the cross section; and/or wherein the one or more nitrogen-vacancy centers or the excitable material radially extend substantially over an entire width of the optical waveguide or extend at least over 50% of the cross section.

27. The remote sensing device of claim 22, wherein the diamond, the one or more nitrogen-vacancy centers, and/or the excitable material is or are arranged at the distal end of the optical waveguide in such a way that at least 0.5% of the secondary light at the distal end can be coupled into the optical waveguide; and/or wherein the one or more nitrogen-vacancy centers and/or the excitable material is or are arranged only over a subregion of a cross section of the distal end of the optical waveguide.

28. The remote sensing device of claim 18, wherein at least one of the following is satisfied:
the optical waveguide has a cross section of between 30 μm and 5000 μm;
the optical waveguide has a length of between 10 mm and 10,000 mm; or
the optical waveguide is configured to be at least partially flexible and/or configured to be at least partially rigid.

29. The remote sensing device of claim 18, wherein at least one of the following is satisfied:
the optical waveguide has a transmission of at least 30% for a wavelength of 532 nm;
the optical waveguide has a transmission of at least 30% for a wavelength in a range of between 600 nm and 800 nm;
the optical waveguide has an attenuation of less than 50 dB/m for a wavelength of 532 nm and/or for a wavelength in a range of between 600 nm and 800 nm;
the optical waveguide is configured as a polarization-maintaining waveguide; or the optical waveguide is configured as a nonmagnetic waveguide.

30. The remote sensing device of claim 18, wherein the optical waveguide comprises at least two different types of structure elements comprising a first type having a first refractive index and a second type having a second refractive index.

31. The remote sensing device of claim 30, wherein the structure elements have a nonuniform arrangement which is defined uniquely by a predetermined rule, the nonuniform arrangement that is defined uniquely by the predetermined rule being configured as at least one of the following:
(a) periodic positioning of structure elements, the periodically positioned structure elements having a variation from one another which is configured nonuniformly but is defined uniquely by a predetermined rule;
(b) aperiodic positioning of structure elements, the aperiodic positions of the structure elements being configured nonuniformly but defined uniquely by a predetermined rule; or
(c) as positioning of structure elements at periodic sites, some of the periodic sites being occupied and some of the periodic sites being unoccupied, and the occupation being configured defined uniquely by a predetermined rule.

32. The remote sensing device of claim 30, wherein the structure elements are arranged in such a way that the optical waveguide has a numerical aperture that is greater than 0.5.

33. A remote sensing unit, comprising:
an optical waveguide having a proximal end and a distal end and adapted for transmission of primary light from the proximal end to the distal end and/or for return transmission of secondary light with a different wavelength, which is induced at the distal end by the primary light, to the proximal end, the optical waveguide having a numerical aperture that is greater than 0.5; and
a light absorption/emission unit arranged at the distal end of the optical waveguide for absorption of the primary light and for emission of the secondary light onto the distal end of the optical waveguide.

34. The remote sensing unit of claim 33, wherein at least one of the following is satisfied:
the optical waveguide is adapted to transmit the primary light and/or the secondary light in a transversely localized fashion with a transverse spatial resolution in such a way that the optical waveguide forms an image guide;
the optical waveguide comprises a multiplicity of structure elements, each of which extends from the proximal end to the distal end and in part over a cross section of the optical waveguide, in such a way that a multiplicity of cross-sectional regions are defined in the cross section of the waveguide, each of which corresponds to the cross section of a single structure element; or
the cross-sectional regions of the structure elements are arranged nonuniformly in order to induce a transverse Anderson localization of the primary light and/or of the secondary light.

35. The remote sensing unit of claim 33, wherein the light absorption/emission unit at the distal end of the optical waveguide comprises a material that enables emission of the secondary light by absorption of the primary light and/or the light absorption/emission unit at the distal end of the optical waveguide comprises an excitable material which has an electronic structure that enables excitation by the primary light and decay by emission of the secondary light.

36. The remote sensing unit of claim 35, wherein at least one of the following is satisfied:
the excitation is enabled by primary light with a wavelength of between 200 nm and 20 μm and/or the decay is enabled by emission of secondary light with a wavelength of between 200 nm and 20 μm; or
the energy states are configured in such a way that an external measurement variable can be measured with the aid of the received secondary light, wherein the external measurement variable is selected from the group consisting of a magnetic field, a conductivity, a temperature, and an amount of substance or substance concentration.

37. An endoscope, comprising:
a remote sensing device or a remote sensing unit, the remote sensing device or the remote sensing unit comprising:
an optical waveguide having a proximal end and a distal end and adapted for transmission of primary light from the proximal end to the distal end and/or for return transmission of secondary light with a different wavelength, which is induced at the distal end by the primary light, to the proximal end, the optical waveguide having a numerical aperture that is greater than 0.5; and
a light absorption/emission unit arranged at the distal end of the optical waveguide for absorption of the primary light and for emission of the secondary light onto the distal end of the optical waveguide.
